(19) United States
(12) Patent Application Publication
(10) Pub. No.: US 2016/0151016 A1
(43) Pub. Date: Jun. 2, 2016

(54) METHODS FOR DEFINING AEROBIC EXERCISE TRAINING ZONES FOR USE IN AN EXERCISE TRAINING PRESCRIPTION AND FOR PROVIDING FEEDBACK ON COMPLIANCE WITH THE PRESCRIPTION

(71) Applicants: Stephen T. Anderson, North Oaks, MN (US); David Anderson, White Bear Lake, MN (US); Bruce D. Johnson, Rochester, MN (US); Dianna Orr, Columbia, MO (US)

(72) Inventors: Stephen T. Anderson, North Oaks, MN (US); David Anderson, White Bear Lake, MN (US); Bruce D. Johnson, Rochester, MN (US); Dianna Orr, Columbia, MO (US)

(21) Appl. No.: 14/308,202

(22) Filed: Jun. 18, 2014

Related U.S. Application Data
(60) Provisional application No. 61/837,464, filed on Jun. 20, 2013.

(51) Int. Cl. A61B 5/00; A61B 5/0205; A63B 24/00

(52) U.S. Cl. CPC A61B 5/4866; A63B 24/0062; A63B 24/0075; A61B 5/0205; A61B 5/4833; A61B 5/486; A61B 5/024

(57) ABSTRACT

A method of pattern recognition for defining aerobic HR training zones measured during incremental cardiopulmonary exercise testing wherein such HR training zones are derived from distinct points in the incremental exercise response for % fat, % CHO, and PetCO₂. Also disclosed is a method for providing feedback to the exercising subject indicating the % time the subject exercised in each of the zones using recorded HR data from each workout.

### CROSS-REFERENCED TO RELATED APPLICATIONS

This application is a non-provisional application of Application No. 61/837,464, filed Jun. 20, 2013 and claims priority from that application which is also deemed incorporated by reference in its entirety in this application.

### STATEMENT REGARDING FEDERALLY SPONSORED RESEARCH OR DEVELOPMENT

Not applicable

### BACKGROUND OF THE INVENTION

I. Field of the Invention

The present invention relates generally to the field of fitness assessment and specifically to a process of identifying exercise heart rates associated with key points observed during a cardiopulmonary exercise test. The present method provides a more sensitive, physiologic, and easier to use method than currently available methods for defining heart rate based aerobic exercise training zones. In addition, the present invention provides feedback on the effectiveness of the prescribed training regimen.

II. Related Art

Exercise training in a fitness club has usually been based upon a subject achieving different heart rate (HR) percentages of his or her age predicted max based upon a table of normals that are for the most part sedentary, such as by the Wasserman database. It is also well known that HR declines with age so that training heart rates at different levels of exertion should follow the age predicted HR ranges. However, what is not taken into consideration is the actual functional state of the subject, which can only be accurately described by direct gas exchange measurements with sub-max evaluation analyzing up to and slightly beyond changes in certain gas exchange metrics that describe or mark the individualized hyperpnoea (respiratory rate acceleration) phase of exercise, when limitations occur in cardiac stroke volume and arterial venous oxygen extraction in the periphery.

The anaerobic threshold (AT) has been long thought of as a point during exercise where metabolic substrate fuel shifts from fats as an energy expenditure contributor to more carbohydrate dominance. It has been thought of as a lower intensity exercise zone whereby an individual can exercise in a steady state for extended periods of time, in comparison to interval zones that are known to be above the AT but slightly below the true max heart rate. However this point of determination, even by direct gas exchange has demonstrated in numerous reference papers that too much variance in user determination exists on the actual AT point, thus creating inaccuracies in its use for training intervals. In addition, the AT is not just a single point but a zone that starts at a cellular level and is then manifested by an “end of zone” point closer to what can now be measured easily by the changes in end expired CO₂.

Errors in the AT detection make it difficult to assess functional improvement beyond the single parameter of VO₂ max.

### SUMMARY OF THE INVENTION

The present advance, to a large extent, obviates the problems discussed in the foregoing in the use of anaerobic threshold and peak VO₂ for determining aerobic exercise training zones. In accordance with the present invention, it has been found that a method that uses substrate utilization and PetCO₂ (end tidal CO₂ partial pressure) is easier to visualize and interpret. Moreover, this method is obtained without exercising the patient to a maximal value, but instead, enables utilization of gas exchange variables commonly measured during submaximal exercise.

The present method of exercise zone-defined training, based upon breath-by-breath cardiopulmonary exercise test data, eliminates the error in determining the AT and relies on the PETCO₂ profile to very easily mark a point in exercise which depicts the onset of respiratory compensation (RC). Likewise, with the use of gas exchange monitoring, intensities of exercise below the interval training zones can be individualized by an easily determined cross over point in fat and carbohydrate utilization for EE, as directly assessed during gas exchange monitoring.

The present invention provides a method for defining an aerobic training regimen in terms of heart rate ranges defining each zone. The delineation of each zone is determined as follows:
1. The point at which PetCO₂ reaches a plateau and subsequently decreases with increasing exercise intensity.
2. The calculation of substrate utilization using the Weir formula using the respiratory exchange ratio (RER)
   a. % fat utilized=((−341.58*RER)+340.37)
   b. % carbohydrate (CHO) utilized=((341.56*RER)−240.36)

In accordance with a preferred method, a cardiopulmonary exercise gas exchange analysis is made for each test data set.

Whereas, the data gathering aspect involves known techniques and analysis, it is aspects of the feature extraction mechanism and the classification scheme from which the invention enables an observer to gain new and valuable insight into the present condition and condition trends in subjects undergoing exercise training.

### BRIEF DESCRIPTION OF THE DRAWINGS

In the drawings:

FIG. 1 is a schematic drawing that illustrates the functional components of a CPX testing system usable with the present invention;

FIG. 2 is a schematic drawing that illustrates one form of exercise protocol that is used to place a volume load on the cardiopulmonary system;

FIG. 3 illustrates an organization of the measured data once it is acquired from the cardiopulmonary exercise gas exchange analyzer;

FIG. 4 illustrates a plot of the Weir equation for RER values in the physiologic range;

FIG. 5 illustrates a plot of the measured variables utilized in the present invention and the individual training zones;

FIGS. 6A and 6B illustrate a training prescription based upon the heart rates determined in FIG. 5;

FIG. 7 illustrates a plot of the uploaded workout heart rates with the heart rate zones overlaid; and

FIG. 8 illustrates a bar graph of the percentage of the total uploaded workout heart rates for each zone.

### DETAILED DESCRIPTION

The following detailed description, including the use of patient data, is intended to be exemplary of a preferred method of utilizing the concepts of the present invention and is not intended to be exhaustive or limiting in any manner with respect to similar methods and additional or other steps which might occur to those skilled in the art. The following description further utilizes illustrative examples, which are believed sufficient to convey an adequate understanding of the broader concepts to those skilled in the art, and exhaustive examples are believed unnecessary.

It is becoming increasingly clear in the fitness markets that individuals are interested in a more scientific approach to exercise training using objective measurements rather than estimates.

The objective information obtained from such testing may prove valuable on several levels including: 1) Assessing the training effect of an exercise prescription, 2) Assessing the compliance with an exercise prescription, and 3) providing goals and incentives to improve the wellness of individuals.

General Considerations—Training Prescription—

The present invention includes a pattern recognition system consisting of a) a cardiopulmonary exercise gas exchange analyzer that gathers the observations to be classified or described, b) a feature extraction mechanism that computes numeric information from the observations, and c) a classification or description scheme that does the actual job of classifying or describing observations based on the extracted features.

Data Gathering:

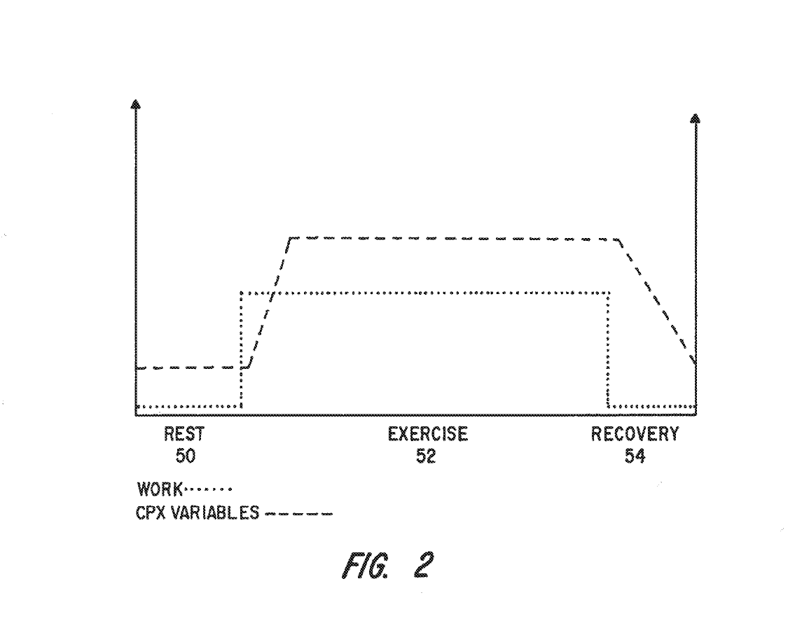

As indicated and shown in FIG. 2, the general class of data utilized in the present invention, cardiopulmonary exercise gas exchange measurements, is obtained 1) at rest, 2) during physical exercise testing performed in accordance with a standardized workload protocol as the forcing function to elicit physiologic changes resulting from the workload, and 3) during a short recovery period following exercise termination. The data measured during exercise quantifies how an individual is able to function in the physical world in terms of the physiologic changes that the individual experiences when engaged in the performance of daily physical work.

The physiologic changes are measured using a cardiopulmonary exercise testing system (CPX) to measure selected variables associated with oxygen consumption, VO₂, carbon dioxide production, VCO₂, end tidal CO₂, PetCO₂, ventilation, VE, respiratory exchange ratio, RER, and heart rate, HR.

As indicated, the data gathering aspect of the invention involves known techniques and analyses, and the calculations for formulating predictive assessments are readily available in the scientific literature. However, by means of aspects of the feature extraction mechanism and the classification scheme, the present invention enables an observer to gain new and valuable insight into the present condition and condition trends in patients. Thus, in accordance with a preferred method, a cardiopulmonary exercise gas exchange analysis is made for each test data set. The performance of such a test is well understood by individuals skilled in the art, and no further explanation of this is believed necessary.

Equipment—

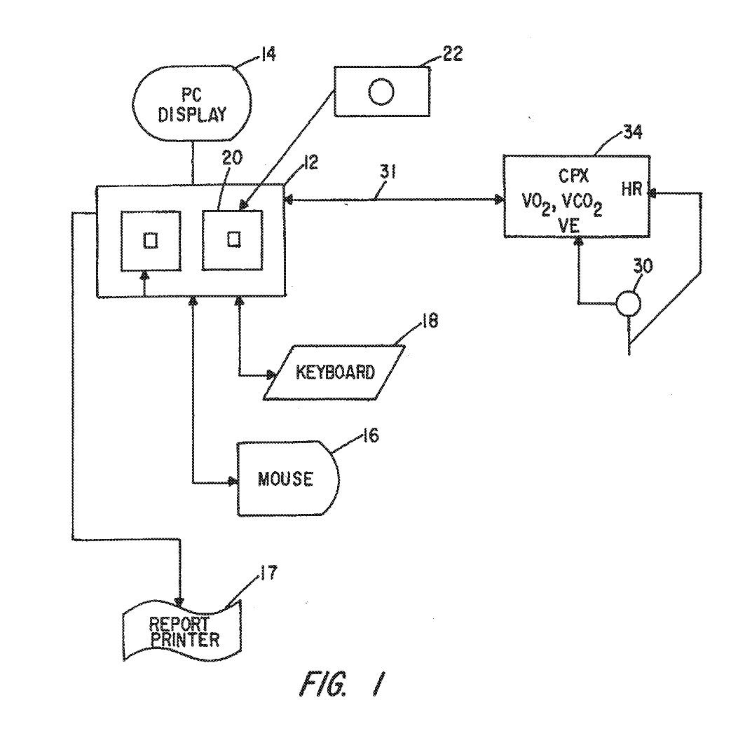

With this in mind typical hardware is shown in FIG. 1, which illustrates typical equipment whereby a cardiopulmonary exercise test (CPX) may be conducted and the results displayed in accordance with the method of the present invention. The system is seen to include a data processing device, here shown as a personal computer of PC 12, which comprises a video display terminal 14 with associated mouse 16, report printer 17 and a keyboard 18. The system further has a floppy disc handler 20 with associated floppy disc 22. As is well known in the art, the floppy-disc handler 20 input/output interfaces comprise read/write devices for reading prerecorded information stored, deleting, adding or changing recorded information, on a machine-readable medium, i.e., a floppy disc, and for providing signals which can be considered as data or operands to be manipulated in accordance with a software program loaded into the RAM or ROM memory (not shown) included in the computing module 12.

The equipment used in the exercise protocol can be a simple stair step of a known height. A CPX testing system 34 interfaces with the subject 30 during operation of the exercise test. The physiological variables may be selected from heart rate (HR), ventilation (VE), rate of oxygen uptake or consumption (VO₂), carbon dioxide production (VCO₂), end tidal CO₂ (PetCO₂), respiratory exchange ratio (RER), or other variables derived from these basic measurements. Physiological data collected is fed into the computing module 12 via a conductor 31, or other communication device.

The workload protocol is illustrated in FIG. 2 and is organized in to a rest phase 50, and exercise phase 52, and a recovery phase 54. Although not required, the workload may also be quantified by requiring the patient to maintain a desired stepping cadence by the addition of an audible metronome that guides the frequency of the steps taken during the exercise phase.

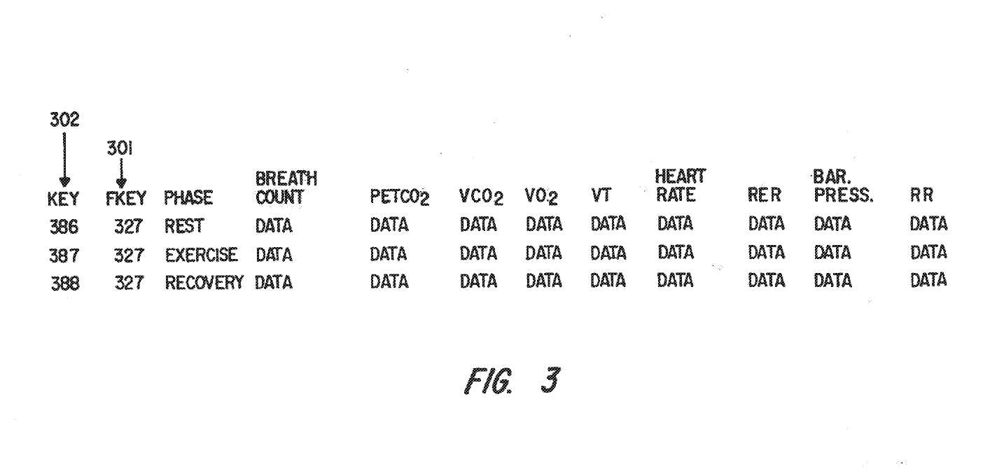

All data acquired by the CPX system is stored in a relational database as illustrated in FIG. 3. Most importantly, data for each patient and each test is stored into separate subsets of data representing the rest phase 386, the exercise phase 387, and the recovery phase 388 for use by the feature extraction mechanism.

Feature Extraction Steps

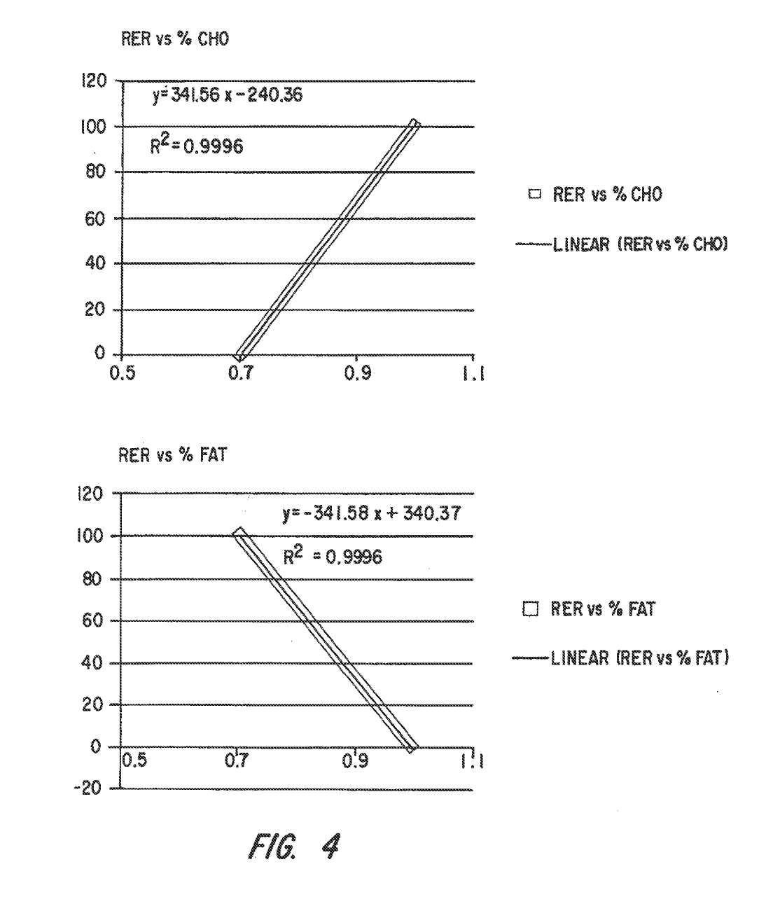

Step 1—The equations for determining substrate utilization have been available for several years, originally by Prof. J. B. de V. Weir in 1948 In Prof. Victor and Frank Katch's textbook (W. McArdle, F. Katch, and V. Katch. Exercise Physiology: Energy, Nutrition and Human Performance; Chapter 8; Measurement of Human Energy Expenditure. 1991 (Third Edition). P 145-158, a table is presented P 153) showing the value of % CHO and % Fat for each value of the RER within the range of human physiology. FIG. 4 shows the data from this table plotted with RER on the x-axis and % CHO and % Fat on the y-axis. When a linear regression analysis is performed on each, the resulting equation is also displayed.

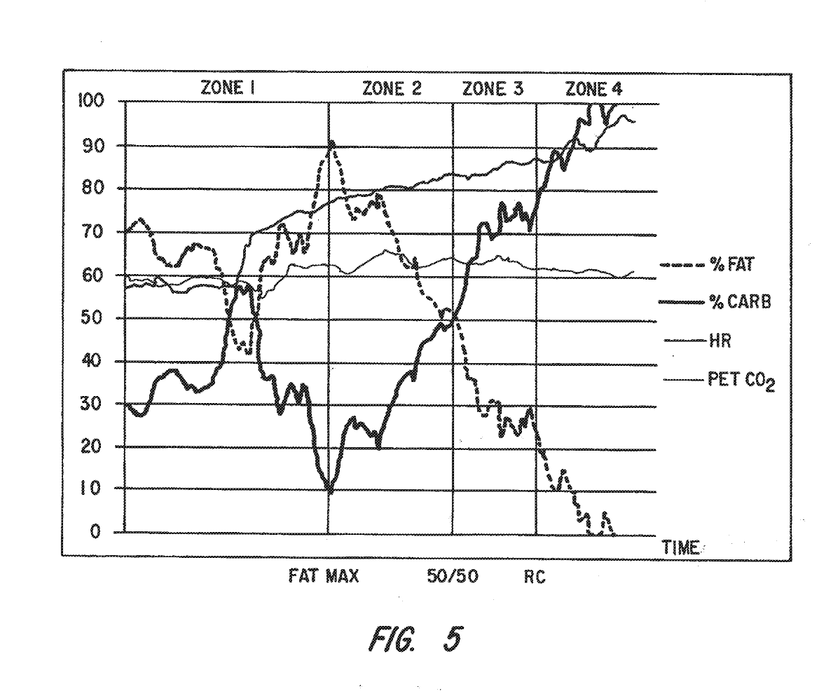

Step 2—In FIG. 5, each of these equations are used to determine the % CHO and % Fat for the measured values of RER for the test. From this data, a line is drawn from the crossover point, where % CHO=% Fat, the line intersecting at the HR value at the same time in the test. This is the first heart rate zone delineation point, and is the easiest criteria for programmatic determination.

Step 3—PetCO₂ plateau—In FIG. 5, the PetCO₂ values measured during the test are displayed. Where this value reaches a peak and starts to decrease (referred to in the scientific literature as the Respiratory Compensation Point, RC), another line is drawn, intersecting again at the HR value at the same time as the test. This is the second heart rate zone delineation point, which can be easily determined programmatically.

Step 4—Fat Max—In FIG. 5, a line is drawn at the peak of the % Fat plot, and this is again intersected with the heart rate value at the same time of the test. In some tests, the Fat Max point will be the same time in the test as the 50/50 time. In this case, there will be only 3 zones instead of the 4 zones identified in FIG. 5.

### Description Scheme

### Zone Training Prescription

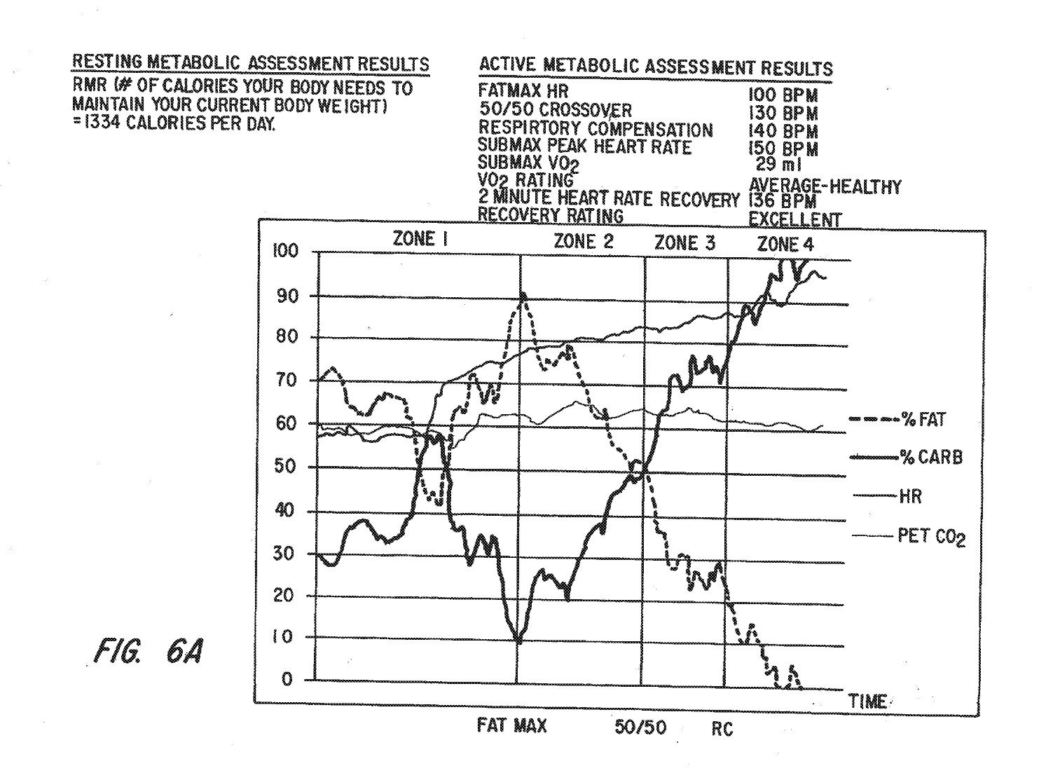

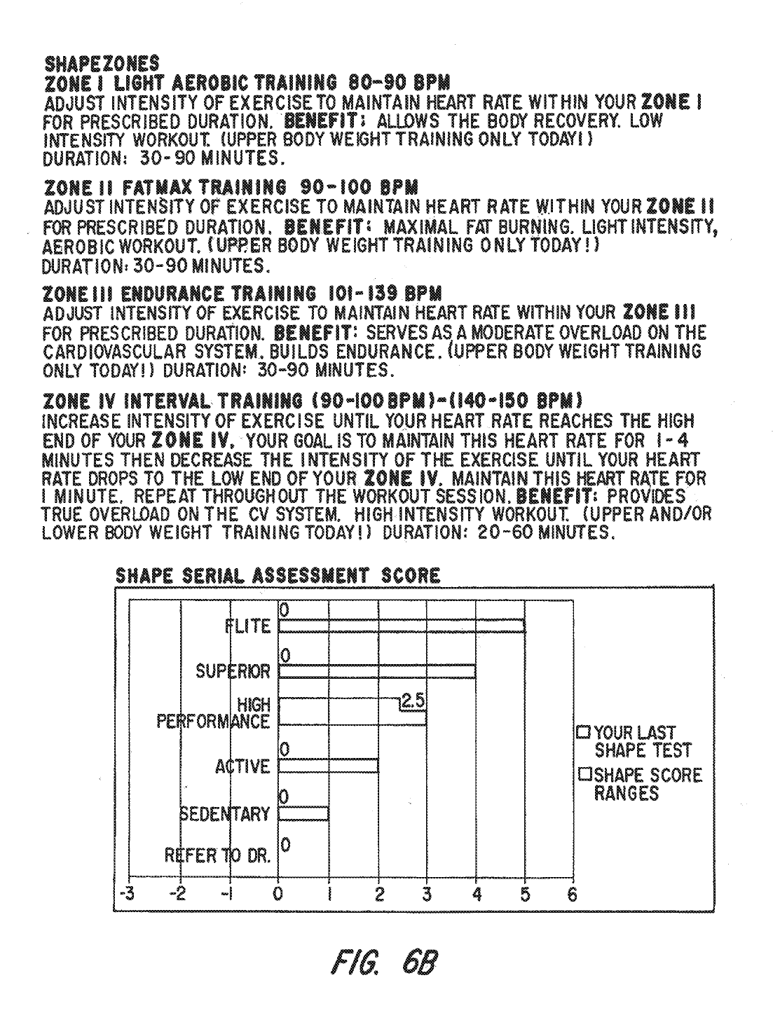

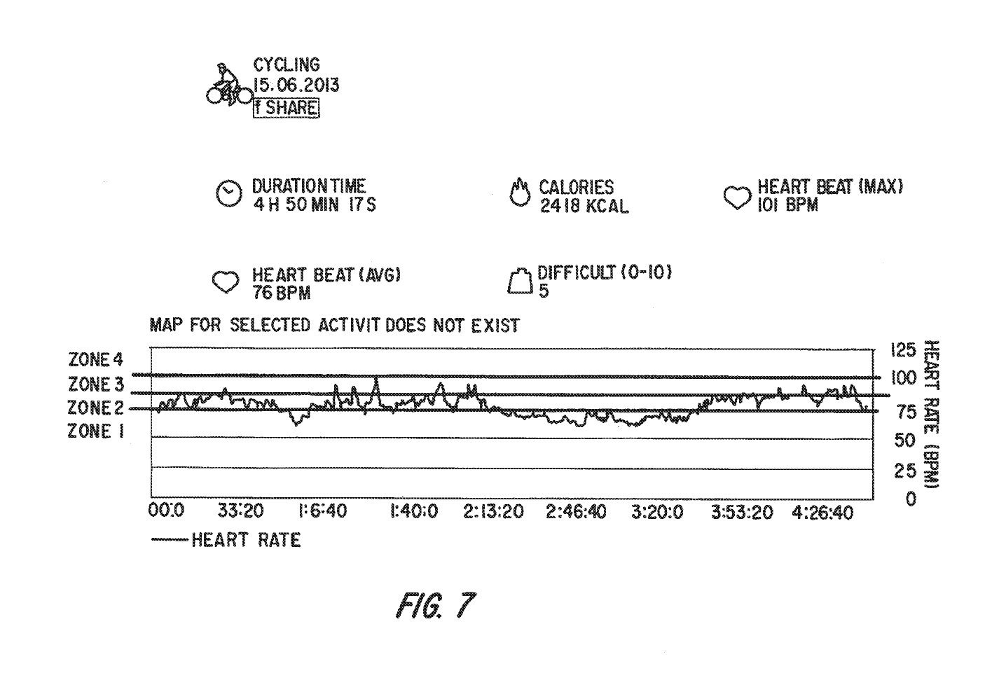

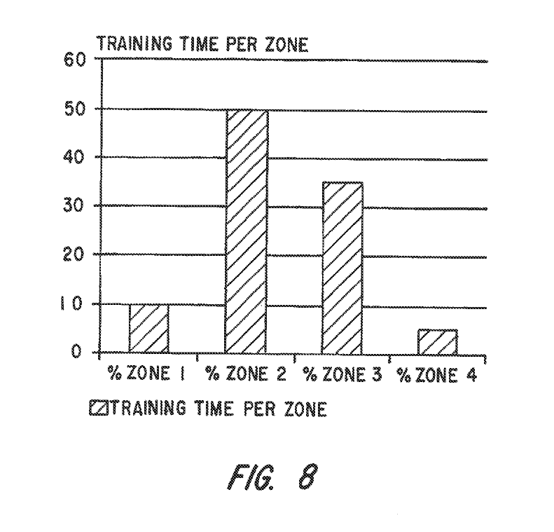

In FIGS. 6A and 6B, subject test data is used in a printed report.

### Description Scheme

### Workout History Report

Several options are available for recording heart rate and other data on wearable wireless devices. Once recorded, the data can be uploaded to a website for display and additional analysis of the uploaded data. One such provider is a-life (a-life.eu.com). In FIG. 7, heart rate date collected during a 5 hour cycling session is plotted. In FIG. 7, the same data is overlaid with heart rate training zones as determined above. In FIG. 8, a bar chart format is illustrated in which the percentage of heart rate data points from one or more workouts are computed as the number of heart rate data points in each zone divided by the total number of heart rate data points for the workout time period (single or multiple workouts).

The invention has been described in considerable detail in order to comply with the Patent Statutes and to provide those skilled in the art with the information needed to apply the novel principles and to construct and use such specialized components as are required. However, it is to be understood that the invention can be carried out by specifically different equipment and devices, and that various modifications, both as the equipment details and operating procedures can be accomplished without departing from the scope of the invention itself.

1. A method for determining exercise heart rate (HR) training zones based upon parameters selected from PetCO₂ and substrate utilization during a cardiopulmonary exercise test wherein substrate utilization is determined by using a respiratory exchange ratio (RER) to determine:
a) % fat utilization (% Fat)=((−341.5*RER)+340.37);
b) % carbohydrate utilization (% CHO))=((341.5*RER)−240.36).

2. A method as in claim 1 wherein the number of zones is 4.

3. A method as in claim 2 wherein the zones are determined by three training zone separation lines located on a plot of % fat utilization, % carbohydrate utilization, PetCO₂ and HR vs time.

4. A method as in claim 3 wherein a first training zone separation line is located at the peak value of the % fat utilization, a second training zone separation line is located where % fat utilization=% carbohydrate utilization and wherein a third training zone separation line is located at the respiratory compensation point (RC) based on PetCO₂ values.

5. A method as in claim 1 wherein a first zone training separation line is located on a plot of % fat utilization, % CHO utilization, and measured heart rate (HR) vs time at the peak value of the % fat utilization plot, the line intersecting at the HR value measured at the same time in the cardiopulmonary exercise test.

6. A method as in claim 1 wherein a second training zone separation line is located on a plot of % fat utilization, % CHO utilization, and HR vs time at the crossover point, where % fat utilization=% CHO utilization, the line intersecting at the HR value measured at the same time in the cardiopulmonary exercise test.

7. A method as in claim 1 wherein a third training zone separation line is located on a plot of PetCO₂ and HR vs time where the value of PetCO₂ reaches a peak and starts to decrease, which is the respiratory compensation point (RC), the line intersecting at the HR value measured at the same time in the cardiopulmonary exercise test.

8. A method as in claim 1 wherein the HR training zones are used to provide an exercise prescription determined by the HR ranges spanning each zone.

9. A method as in claim 8 wherein the number of zones is 4.

10. A method as in claim 9 wherein the zones are determined by three training zone separation lines located on a plot of % fat utilization, % carbohydrate utilization, PETCO₂ and HR vs time.

11. A method as in claim 10 wherein a first training zone separation line is located at the peak value of the % fat utilization, a second training zone separation line is located where % fat utilization=% carbohydrate utilization and wherein a third training zone separation line is located at the respiratory compensation point (RC).

12. A method for providing feedback on compliance to an exercise prescription whereby HR data recorded during an aerobic training session are computed and displayed in graphical and/or numeric form as the number of heart rate data points in each HR training zone divided by the total number of HR data points for the workout or workouts.

13. A method as in claim 12 wherein the aerobic training session HR data is compiled through a plurality of training zones which are based upon parameters selected from PetCO₂ and substrate utilization during a cardiopulmonary exercise test wherein substrate utilization is determined by using a respiratory exchange ratio (RER) to determine:
a) % fat utilization (% Fat)=((−341.5*RER)+340.37);
b) % carbohydrate utilization (% CHO))=((341.5*RER)−240.36).
